(19) United States
(12) Patent Application Publication
(10) Pub. No.: US 2024/0425554 A1
(43) Pub. Date: Dec. 26, 2024

(54) PRODUCTION OF PLANT PROTEIN ISOLATES EMPLOYING GLUTAMINASE ENZYME

(71) Applicant: CORN PRODUCTS DEVELOPMENT, INC., WESTCHESTER, IL (US)

(72) Inventors: Hanyu YANGCHENG, Bridgewater, NJ (US); Matthew HENLEY, Bridgewater, NJ (US)

(21) Appl. No.: 18/693,422

(22) PCT Filed: Oct. 6, 2022
(86) PCT No.: PCT/US2022/045835

§ 371 Date: Mar. 19, 2024

Related U.S. Application Data
(60) Provisional application No. 63/257,707, filed on Oct. 20, 2021.

(51) Int. Cl. C07K 14/415; C07K 1/34; C12N 9/80

(52) U.S. Cl. CPC C07K 14/415; C07K 1/34; C12N 9/80; C12Y 305/01002

(57) ABSTRACT

This specification discloses methods for isolating a plant protein using a glutaminase enzyme. The methods comprise soaking a proteinaccous plant based starting material, in an aqueous solution comprising at a temperature less than about 45° C. less than about 5 hours to obtain a deamidated plant protein then separating the deamidated plant protein from the aqueous solution drying the deamidated plant protein.

The technology disclosed in this specification pertains to methods for making plant protein isolates. More specifically, the disclosed methods use enzymatic deamidation to obtain a plant protein isolate having a specific deamidation profile.

Isolated proteins are useful food ingredients. They can be used in food compositions, which, in this specification, includes beverages. The protein may be used to provide one or more of nutrition, structure, or functional performance to the food composition. Proteins selected for their functional performance, commonly suffer from changes in their functional performance over time. On class of functional attributes of interest for protein isolates is their ability to dissolve in aqueous solutions. It has been observed, however, that the dissolution properties of protein-isolates commonly decrease over time, and so the protein isolates become less functional. Methods for preparing protein isolates that can stabilize their functionality over time are desirable.

This specification discloses a method to extract deamidated plant protein from plant flour or plant protein concentrate. This specification also discloses deamidated plant protein isolates having stable dissolution profiles compared to non-deamidated plant protein isolates. This specification also discloses food compositions comprising the disclosed plant protein isolates.

Deamidated pea proteins and methods of making them from an isolated pea protein are described in International Patent Publication WO 2021-236749A1, which is incorporated in this specification in its entirety. The '749 publication also describes functional attributes of deamidated pea protein isolates as well uses of deamidated pea protein isolates in food products. Particularly, in cold pressed bars and beverage powder mixes. This specification extends on the disclosure of the '749 publication by describing how to obtain legume protein isolates, including pea protein isolates, having the preferred degrees of deamidation describe by using the glutaminase enzyme to isolate protein from a legume flour or pea flour.

In any embodiment a plant protein isolate is obtained from proteinaceous plant material. Examples include whole seeds, split seeds, whole tubers, split tubers, flaked tubers, milled plant compositions such. Examples of milled plant compositions include flours, or high protein flours, low protein flours. Within this specification flours may be obtained by any suitable means including dry milling or wet milling. Also dry milled or wet milled flours may be further process using air classification to increase or reduce the relative protein content within the flour. Suitable plant materials include but are not limited to, legumes, which include but are not limited to peas, lentils, fava beans, and chickpeas. Other suitable materials include grains such as, but not limited to corn, rice, oats, wheat, and quinoa. Still other suitable materials include tapioca or potato. In more preferred embodiments the proteinaceous plant material is quinoa, or pea, or fava bean, or lentil, or chickpea. In at least some embodiments the proteinaceous plant material is pea.

In any embodiment described in this specification, a method for obtaining a plant protein isolate comprises soaking proteinaceous starting material, preferably legume (such as pea or fava bean or chick pea) starting material in an aqueous solution comprising from about 0.01 to about 0.25% glutaminase enzyme (wt. % relative to the starting material); at a temperature less than about 45° C., or less than about 40° C., or from about 20° C. to about 40° C., or from about 30° C. to about 40° C. for time of less than about 5 hours or less than about 4 hours or from about 0.1, or from 1, or from 2 or from 3 to about 4 hours to obtain a deamidated plant protein solution, optionally, adjusting the pH of the solution to a pH where the deamidated plant protein is least soluble in water separating precipitated plant protein from the remainder of the solution using a filtering or separation technique (like centrifugation) and drying the collected deamidated plant protein. In at least some embodiments the proteinaceous starting material is soaked in an aqueous solution having a pH from about 6 to about 8 or from about 6.5 to about 7.5 prior to or as part of the deamidation process.

In embodiments using a plant flour or legume flour or pea flour or fava bean flour, or chickpea flour as a starting material, the flour is added to water to make slurry of 1-20% solids. Glutaminase may be added to the water prior to or after adding the flour. Deamidation is done by mixing flour water and glutaminase well to form a uniform mixture.

Following deamidation, glutaminase enzyme can be deactivated by increasing the temperature of the slurry to at least about 80° C. for at least about 10 minutes. While in some embodiments deamidated plant protein is recovered from slurry by pH adjustment. The deamidated plant protein can be recovered from solution using any useful method. Such methods include but are not limited to precipitation or flocculation of the deamidated protein from solution followed by centrifuging or filtration. In other embodiments, deamidated plant protein isolates may also be recovered from solution without use of a precipitation step by using evaporative techniques known in the art including spray drying or freeze drying.

In any embodiment described in this specification a deamidated plant protein isolate is obtained by soaking a proteinaceous plant-based material selected from the group consisting of whole grain, whole seed, polished grain, split seed, and flour. In some embodiments glutaminase enzyme is added whole grain, whole seed, polished grain (dehulled, debranned, or both), or split seed, which is then milled to separate the protein from other components.

In any embodiment described in this specification, a method for making a deamidated plant protein isolate comprises separating a deamidated pea protein by adjusting the pH of an aqueous solution comprising the deamidated plant protein to a pH from about 4.0 to about 4.5.

In any embodiment described in this specification, a method for making a deamidated plant protein isolate comprises separating a deamidated pea protein by adding calcium (or calcium containing compound) to the aqueous solution to “salt-out”—i.e. using the salt to alter the solubility of the solution. Illustrative calcium containing compounds include for example, but are not limited to calcium hydroxide, calcium carbonate, and calcium chloride.

In any embodiment the separation step comprises doing one or more of adjusting the pH of an aqueous solution comprising a deamidated plant protein to from about to about 4.5 or adding calcium to the aqueous solution and then using filtration or centrifugation before and/or after the pH adjustment and/or adding of calcium to remove at least part of the deamidated plant protein from the aqueous solution.

In any embodiment of the method described in this specification, the deamidated protein isolated obtained by the method has a yield (isolate recovered/protein in base flour×100) greater than 45% or from about 45% to about 75% or from about 45% to about 70%, or from about 45% to about 65%, or from about 50% to about 75%, or from about 55% to about 70%, or from about 55% to about 65%, or from about 60% to about 75% or about 60% to about 70%, or from about 60% to about 65%.

In any embodiment of the method described in this specification, the deamidated protein isolate obtained by the method has a purity (protein content of the isolate wt. %) of greater than about 85% or from about 85% to about 95% or from about 85% to about 90%.

In another aspect, the technology disclosed in this specification pertains to a deamidated plant protein isolate. The plant protein isolates can be obtained by the methods described in this specification. Plant protein exists in plants in amounts various amounts. In any embodiment a plant protein isolate has protein content of greater than about 70%, or greater than about 75%, or from about 75% to about 90% or from about 75% to about 85% protein by weight and has a degree of deamidation between about 10% and about 20%, or about 12% to about 20% or about 15% to about 20%.

Advantageously the deamidated plant protein isolates described in this specification are more water soluble than plant protein isolates obtained from the same base material that are not deamidated or otherwise modified following isolation. Solubility of the deamidated plant protein isolate varies with pH. In embodiments of the described deamidated plant protein isolates, the solubility of the isolate is as follows. In any embodiment described in this specification, a deamidated plant protein isolate has a percent soluble material content at pH 6 of greater than 30% or greater than 35%, or from about 30% to about 50%, or from about 35% to about 50%. In any embodiment described in this specification, a deamidated plant protein isolate has a percent soluble material content at pH 5 of greater than about 30% or from about 30% to about 40%. In any embodiment described in this specification, a deamidated plant protein isolate has a percent soluble material content at about pH 7 of greater than about 40% or from about 40% to about 50%

In any embodiment described in this specification, a deamidated plant protein isolate has a median particle size of between 75 and 125 microns, or from about 80 to about 120 microns, or from about 90 to about 100 microns.

In any embodiment described in this specification, a deamidated plant protein isolate swells less in the presence of water and has lower numbers of large particles than non-deamidated plant proteins isolates. In any embodiment described in this specification, a deamidated plant protein isolate comprises a set of particles that, when measured for size in an aqueous slurry, has a particle size distribution, the particle size distribution comprising a set of particles having a size between 150 and 300 microns, wherein a peak number of particles between 150 and 300 microns is less than about 30, or less than about 25, or less than about 20, or less than about 15. Within this specification, a particle size measurement useful for determining the peak number of particles having size between 150 and 300 microns is as follows: a slurry of plant protein isolate in water (5% solids in water w/w) is continuously mixed over period of at least 10 minutes, and the particle size distribution is measured using focused beam reflectance.

In any embodiment described in this specification, a deamidated plant protein isolate has a set of particles that, when measured for size in an aqueous slurry, have a particle size distribution, the particle size distribution comprising a set of particle having a size between 150 and 300 microns, wherein the slurry is measured to have less than about 15 particles having a size between 150 and 300 microns, or less than about 10. Within this specification, a particle size measurement useful for determining the number of particles having size between 150 and 300 microns in slurry is measured in a process consisting of adding plant protein isolate 10 water to form a slurry having 5% solids (w/w) and measuring the particle size distribution immediately following adding the protein.

In any embodiment described in this specification, a deamidated plant protein isolate has an equal or an improved solubility index compared to a fresh sample of a deamidated plant protein isolate. In any embodiment described in this specification, a deamidated plant protein is made by a process comprising storing the deamidated plant protein isolate for at least about 1 month, or at least about 3 months, or at least about 6 months, or at least about 1 year. In any embodiment described in this specification, a deamidated plant protein isolate is obtainable by a process comprising storing the deamidated plant protein isolate at a temperature of from about 30° to about 50° C., or from about 35° C. to about 45° C., or from about 38° C. to about 42° C. for a time of from about 15 to about 45 days, or from about 20 to about 40 days, or from about 25 to about 35 days, or about one month. In any embodiment described in this specification, a deamidated plant protein isolate made by a process comprising storing the deamidated plant protein isolate at a temperature of from about 30° to about 50° C., or from about 35° C. to about 45° C., or from about 38° C. to about 42° C. for a time of from about 15 to about 45 days, or from about 20 to about 40 days, or from about 25 to about 35 days, or about one month.

In any embodiment of a deamidated plant protein isolate described in this specification, the deamidated plant protein isolate has a degree of hydrolysis of less than 10%, or less than 5%, or essentially 0% as measured by SDS-PAGE comparing deamidated and non-deamidated plant protein isolate.

In another aspect, the technology disclosed in this specification pertains to the use of a deamidated plant protein isolate in a food composition. In any embodiment, a food composition as disclosed in this specification comprises a deamidated plant protein isolate and a second edible ingredient. In any embodiment, a food composition is a powdered composition comprising a deamidated plant protein isolated and a second powdered ingredient. In any embodiment, a food composition has at least about 15% deamidated plant protein isolate (w/w of the powder), or at least about 25% or at least about 50%, or at least about 60%, or at least about 70%.

In any embodiment, a food composition comprising a deamidated plant protein isolate described in this specification further comprises any second ingredient commonly used in food compositions. In any embodiment, of a food composition described in this specification includes a starch including but not limited to corn starch, tapioca starch, pea starch, fava bean starch, lentil starch, chickpea starch, tapioca starch, potato starch, and sago starch as well as high amylose and low amylose variants of such starches. Such starches also may be within flours and meals including wheat flour and nut meals. Useful starches may be modified or unmodified. Modified starches may be crosslinked including by using phosphate or adipate, or may be stabilized, including hydroxypropylated and acetylated. Useful starch may be converted or hydrolyzed using shear, enzyme, acid, or oxidation. Starch may also be modified usefully by oxidation for purposes other than hydrolysis. Useful starch may be physically modified such as by thermal inhibition, annealing, or heat moisture treatments. Modified and unmodified starch may be pregelatinized or otherwise made cold water soluble.

In any embodiment, a food composition comprising a deamidated plant protein isolate described in this specification may further comprise a sweetener. Useful sweeteners include honey, allulose, tagatose, fructose, glycerol, sucrose, rebaudiosides (A, B, J, M, etc.), and glucosylated stevia glycosides, corn syrups including high fructose corn syrups. Sweeteners may be provided in solid, or powdered, or liquid, or syrup form.

In any embodiment, a food composition comprising a deamidated plant protein isolate described in this specification may further comprise a fiber. Useful fibers may include cellulosic fibers from any botanical source, resistant starches, soluble fibers such as polydextrose or short chain fructooligosacchardies.

In any embodiment, a food composition comprising a deamidated plant protein isolate described in this specification may further comprise a gum or gum-like material. Useful gums and gum like materials include gelling starches, gum Arabic, xanthan gum, tara gum, konjac, carrageenan, locust bean gum, gellan gum, guar gum, pectin, and modified celluloses like carboxymethyl cellulose, and mixtures thereof.

In any embodiment, a food composition comprising a deamidate plant protein isolate described in this specification may further comprise an oil, or fat, or aqueous ingredient. Useful oils include vegetable oils such as corn oil, olive oil, canola oil, sunflower oil, rapeseed oil, palm oil, coconut oil. Useful fats (other than vegetable oils) included animal fats and dairy fats. Useful aqueous ingredients include water, milk, syrups, or other carbohydrate containing liquids, or acidic liquids, or basic liquids.

In any embodiment, a food composition comprising a deamidated plant protein isolate described in this specification may further comprises various other flavorings and coloring commonly used in food composition.

In any embodiment, a food composition, as described in this specification, is a beverage or a cold pressed bar, or powder useful for making a beverage.

In any embodiment, this specification discloses a food composition that is a beverage comprising a deamidated plant protein isolate and an aqueous liquid. In any embodiment, a beverage, as disclosed in this specification is made from a powdered mix comprising a deamidated plant protein isolate. In any embodiment, this specification discloses a beverage comprising a deamidated plant protein isolate and has a pH of less than about 7, or from about 7 to about 2. In any embodiment, this specification discloses a beverage having a pH from about 5 to about 7, or from about 6 to about 7. In any embodiment, this specification discloses a beverage comprising a deamidated plant protein isolate and has a pH of from about 2 to about 4, or about 2 to about 3.5, or from about 2 to about 3.

In any embodiment, this specification discloses a beverage comprising a deamidated plant protein isolate made by a process comprising mixing an aqueous liquid and a powdered composition. In any embodiment, this specification discloses a beverage comprising a deamidated plant protein isolate made by a process comprising mixing an aqueous liquid with a powdered composition having a deamidated plant protein isolate and a second soluble ingredient. In any embodiment, this specification discloses a beverage made from a powdered composition comprising a deamidated plant protein isolate in an amount of at least about 25%, or at least about 50%, or at least about 60%, or at least about 70%, or from about 70% to about 80%. In any embodiment disclosed in this specification, a beverage made from a powdered composition comprising a deamidated plant protein isolate has a protein content of at least about 25%, or at least about 50%, or at least about 60%, or at least about 70%. In any embodiment disclosed in this specification, a beverage made from a powdered composition comprising a deamidated plant protein isolate has a protein content of at least about 25%, or at least about 50%, or at least about 60%, or at least about 70% wherein the protein comes solely from a deamidated plant protein isolate.

In any embodiment, this specification discloses a beverage made by mixing a powder comprising a deamidated plant protein and an aqueous liquid has a peak number of particles having size between 150 and 300 microns that is less than about 30, or less than about 25, or less than about 20, or less than about 15.

In any embodiment, this specification describes a powdered mix for making a beverage wherein a test beverage consisting of 15% (w/w) of the powdered mix in water has a peak number of particles having size between 150 and 300 microns that is less than about 30, or less than about 25, or less than about 20, or less than about 15; wherein the peak particle size is measured using focused beam reflectance while continuously mixing the beverage for at least 10 minutes.

In any embodiment, this specification describes a powdered mix for making a beverage wherein the mix has a soluble material content of at least about 20%, or at least about 25%, or at least about 30% or at least about 35% of the powdered composition is soluble in the aqueous liquid.

In any embodiment, this specification describes a cold pressed food bar comprising a deamidated plant protein isolate and an aqueous ingredient wherein, the cold pressed food bar has a solids content of greater than about 60% (w/w of the bar) or greater than about 70%, from about 60% to about 95%. In any embodiment, an aqueous ingredient for using in a cold pressed food bar, as described in this specification is binding agent, and may be syrup, or other carbohydrate containing liquid. In any embodiment, this specification describes a cold pressed food bar having deamidated plant protein isolate in an amount from 15% to about 50% (w/w of the bar), or from about 15% to about 40%, or from about 15% to about 30%, or from about 20% to about 30%.

In any embodiment, this specification describes a cold pressed food bar comprising a deamidated plant protein isolate that has been deamidated to a degree to control the solubility of the plant protein isolate. In any embodiment described in this specification, a deamidated plant protein isolate for use in a cold pressed food bar has a degree of deamidation of from between about 10% and about 15%, or about 12% and about 14%. In any embodiment described in this specification, a deamidated plant protein isolate for use in a cold pressed food bar has a degree of deamidation of between about 17% and about 23%, or between about 19% and about 21%. In any embodiment described in this specification, a cold pressed food bar has a soluble material content at about pH 7 of between about 30% and about 36%, or about 33% and about 36 (wt %).

In any embodiment a deamidated protein comprises an aqueous ingredient, for example a syrup in an amount of from about 25% to about 35% by weight of the bar.

In any embodiment, this specification describes a cold pressed food bar comprising a deamidated plant protein isolate wherein the degree of deamidation is controlled to reduce the hardness of the cold pressed food bar over time. In any embodiment, a cold pressed food bar described in this specification has a mechanical hardness of less than about 2000 g, or less than about 1500 g. In any embodiment, a cold pressed food bar described in this specification has a mechanical hardness of less than about 2000 g, or less than about 1500 g after at least 1 month's storage, or 2 months' storage, or at least 3 months' storage, or at least 6 months' storage. In any embodiment described in this specification, a cold pressed food bar and has a percent increase in hardness over 1-, 2-, 3-, or 4-months storage at ambient temperature of less than 40%.

The subject matter described in this specification can be better understood with reference to the following definitions and guidance for construing the terms in this specification.

Reference to the term “plant protein isolate” within this specification means a composition that substantially comprises plant protein. Commonly plant protein isolates are obtained from milled plant compositions (e.g. plant flour) and the plant protein within the flour is increased relative to other using methods, such as those described in this specification, by removing other components of the flour, like starch and fiber. In preferred embodiments described in this specification, a plant protein isolate has protein content greater than about 70%, or greater than about 75%, or from about 75% to about 90% or from about 75% to about 85% protein by weight. The rest of the plant protein isolate is comprised substantially of plant starch and plant fiber (cellulosic material).

Reference to the term “deamidated plant protein isolate” in this specification means a plant protein isolate that has been subjected to a process that removes free amine groups from amino acid residues (glutamine residues) in plant proteins of the plant protein isolate. Preferably, (but not necessarily) deamidated plant protein isolates as described are not further modified, for example by hydrolysis or other enzymatic action (like crosslinking using transglutamiase).

Reference to the term “degree of deamidation” in this specification measures amount of ammonia released during deamidation reaction compared to the total ammonia of releasable by the protein. Within this specification, degree of deamidation is reported as a percentage.Degree of deamidation can be calculated using any suitable method in the art. A useful test for measuring degree of deamidation follows. Calculated total ammonia released as follows. Mix three volumes of the enzyme reaction slurry with 1 volume of 40% trichloroacetic acid (“TCA”) solution to obtain mixture having a final TCA concentration of 10%. Centrifuged at 4,000 g for 10 min. Free ammonia content in the supernatant is determined using a commercial ammonia analysis kit (Megazyme). Calculated total ammonia obtainable from the unmodified plant protein isolate as follows. Incubate the unmodified plant protein isolate in 2M H₂SO₄ solution at boiling temperature (about 100° C.) for 2 hours. After the reaction, mixed 3 volumes of the slurry with 1 volume of 40% TCA solution to obtain a mixture having a final TCA concentration of 10%. Centrifuge the mixture at 4,000 g for 10 min. Free ammonia content in the supernatant was determined using the Megazyme ammonia analysis kit. Degree of deamidation equals total ammonia release divided by total ammonia obtainable multiplied by 100.

Within this specification dissolution properties of deamidated protein are analyzed in part by reference to a test that disperses plant protein in a water (5% solids in water w/w), mixes the protein in water using a common low speed stirring device such as a magnetic stirrer suitable for allowing particle size measurements using focused beam reflectance.

Reference to the term “peak number of particles” in this specification refers to largest number of particles observed within all or part a distribution of particles of a composition that is dispersed in water, like a dispersion of plant protein isolate. A useful test for measuring peak particle size of a composition dispersed in water measures the particle size distribution of the composition for least 10 minutes of continuous stirring.

Use of “about” to modify a number is meant to include the number recited plus or minus 10%. Where legally permissible recitation of a value in a claim means about the value. Use of about in a claim or in the specification is not intended to limit the full scope of covered equivalents.

Recitation of the indefinite article “a” or the definite article “the” is meant to mean one or more unless the context clearly dictates otherwise.

While certain embodiments have been illustrated and described, a person with ordinary skill in the art, after reading the foregoing specification, can effect changes, substitutions of equivalents and other types of alterations to the methods, and of the present technology. Each aspect and embodiment described above can also have included or incorporated therewith such variations or aspects as disclosed regarding any or all the other aspects and embodiments.

The present technology is also not to be limited in terms of the aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to methods, conjugates, reagents, compounds, compositions, labeled compounds or biological systems, which can, of course, vary. All methods described herein can be performed in any suitable order unless otherwise indicated herein or otherwise clearly contradicted by context. It is also to be understood that the terminology used herein is for the purpose of describing aspects only and is not intended to be limiting. Thus, it is intended that the specification be considered as exemplary only with the breadth, scope and spirit of the present technology indicated only by the appended claims, definitions therein and any equivalents thereof. No language in the specification should be construed as indicating any non-claimed element as essential.

The embodiments illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the technology. This includes the generic description of the technology with a proviso or negative limitation removing any subject matter from the genus, regardless of whether the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member, and each separate value is incorporated into the specification as if it were individually recited herein.

The technology disclosed in this specification can be better understood with regard to the following aspects which are not intended to limit the full scope of inventions disclosed in this specification.

A method of isolating a plant protein comprising: a) soaking a proteinaceous plant based starting material, in an aqueous solution comprising from about 0.01 to about 0.25% glutaminase enzyme (wt. % relative to the starting material) at a temperature less than about 45° C., or less than about 40° C., or from about 20° C. to about 40° C. for time of less than about 5 hours or less than about 4 hours or from about 0.1, or from 1, or from 2 or from 3 to about 4 hours; b) separating the deamidated plant protein from the aqueous solution; c) and drying the deamidated plant protein. wherein optionally, the aqueous solution in step a) has a pH from about 6 to about 8, or from about 6.5 to about 7.5 to deamidate a plant protein in the proteinaceous plant based starting material.

The method of claim 1 wherein the proteinaceous plant-based material is selected from the group consisting of whole grain, whole seed, polished grain, split seed, and flour; wherein if the proteinaceous plant-based material is whole grain, whole seed, polished grain, or split seed, the method further comprises milling the whole grain, whole seed, polished grain, or split seed prior to the separating step; wherein, optionally, the glutaminase enzyme is added to whole grain, whole seed, polished grain, or split seed prior to milling.

The method of claim 1 or 2 wherein the proteinaceous plant-based material is selected from the group consisting of fava bean, pea, lentil, chickpea, and quinoa.

The method of any one of claims 1 to 3 wherein the proteinaceous plant-based material is pea.

The method of any one of claims 1 to 3 wherein the separating step comprises adjusting the pH of the aqueous solution to from about 4.0 to about 4.5.

The method of any one of claims 1 to 4 wherein the separating step comprises adding calcium to the aqueous solution.

The method of claim 6 or 7 wherein the separating step comprises adjusting the pH of the aqueous solution to from about 4.0 to about 4.5.

The method of any one of claims 6 to 8 wherein separating step comprises adding calcium to the aqueous solution.

The method of any one of claims 1 to 8 further comprising a step following step a) comprising inactivating the glutaminase enzyme by increasing the temperature the aqueous solution comprising the pea flour to a temperature of at least about 80° C. for at least about 10 minutes.

The method of any one of claims 1 to 9 wherein the separating step comprises, applying one or more centrifugation or filtration to separate at least a portion of the deamidated plant protein from the aqueous solution and then doing one or both of adjusting the pH of the aqueous solution to from about 4.0 to about 4.5 and adding calcium to the aqueous solution

The method of any one of claims 1 to 10 wherein the separating step comprises doing one or both of adjusting the pH of the aqueous solution to from about 4.0 to about 4.5, and adding calcium to the aqueous solution, and then applying one or more of centrifugation or filtration to separate at least a portion of the plant protein from the aqueous solution.

The method of any one of claims 1 to 11 wherein the separating step doing one or both of adjusting the pH of the aqueous solution to from about 4.0 to about 4.5, and adding calcium to the aqueous solution, and using one or more of filtration before and after the pH adjustment or calcium addition.

The method of any one of claims 1 to 12 wherein the deamidated plant protein is a deamidated pea protein having a degree of deamidation of between about 10% and about 20%, or about 12% to about 20% or about 15% to about 20% (compared to a base pea protein).

The method of any one of claims 1 to 13 wherein the deamidated plant protein is a deamidated pea protein having a percent soluble material content at pH 6 of from about 20% to about 25%, or between about 20% and about 24%, or between about 20% and about 23% (wt%).

The method of any one of claims 1 to 14 wherein the deamidated plant protein is a deamidated pea protein having a percent soluble material content at pH 2 of between about 15% and about 20%, or about 17% and 20% (wt%).

The method of any one of claims 1 to 15 wherein the deamidated plant protein is a deamidated pea protein having a set of particles that, when measured for size in a slurry, have a particle size distribution, the particle size distribution comprising a set of particles having a size between 150 and 300 microns, wherein a peak number of particles between 150 and 300 microns is less than about 30, or less than about 25, or less than about 20, or less than about 15; and wherein the peak number of particles between 150 and 300 microns in the slurry is measured in a process consisting of adding pea protein isolate in water to form a slurry having 15% solids and measuring the particle size distribution for at least 10 minutes using focused beam reflectance while continuously mixing the slurry during the measuring.

The method of any one of claims 1 to 16 wherein the deamidated plant protein is a deamidated pea protein having a set of particles that, when measured for size in a slurry, have a particle size distribution comprising a set of particles having a size between 150 and 300 microns, wherein the particle size in slurry is measured in a process consisting of adding pea protein isolate in water to form a slurry having 15% solids and measuring the particle size distribution immediately following adding the protein; and wherein the slurry is measured to have less than about 15 particles having a size between 150 and 300 microns, or less than about 10.

The method of any one of claims 1 to 17 wherein the deamidated plant protein is a deamidated pea protein that when dispersed in water in an amount of 15% solids (wt. %) forms a slurry such that when the slurry is continuously mixed for at least 10 minutes, the slurry has a particle size distribution comprising a set of particles having a size between 150 and 300 microns, wherein a peak number of particles between 150 and 300 microns is less than about 30, or less than about 25, or less than about 20, or less than about 15.

The method of any one of claims 1 to 18 wherein the deamidated plant protein is a deamidated pea protein having a particle size distribution immediately following adding the protein to the water is measured to have less than about 15 particles having a size between 150 and 300 microns, or less than about 10.

The method of any foregoing claim having a yield (isolate recovered/protein in base flour×100) greater than about 60% or from about 60% to about 65%.

The method of any foregoing claim wherein the method has a recovery efficiency of at least about 90% or at least about 92%, or at least about 94%.

A method of isolating a plant protein comprising: soaking a proteinaceous plant based starting material, in an aqueous solution comprising from about 0.01 to about 0.25% glutaminase enzyme (wt. % relative to the starting material) at a temperature less than about 45° C., or less than about 40° C., or from about 20° C. to about 40° C. for time of less than about 5 hours or less than about 4 hours or from about 0.1, or from 1, or from 2 or from 3 to about 4 hours; drying the deamidated plant protein wherein, optionally, the aqueous solution in step a) has a pH from about 6 to about 8, or from about 6.5 to about 7.5 to deamidate the plant protein.

The method of claim 22 wherein the proteinaceous plant-based material is selected from the group consisting of whole grain, whole seed, polished grain, split seed, and flour; wherein if the proteinaceous plant-based material is whole grain, whole seed, polished grain, or split seed, the method further comprises a step to mill the whole grain, whole seed, polished grain, or split seed prior to the precipitating step; wherein, optionally, the glutaminase enzyme is added to whole grain, whole seed, polished grain, or split seed prior to milling.

The method of claim 22 or 23 wherein the proteinaceous plant-based material is selected from the group consisting of fava bean, pea, lentil, chickpea, and quinoa.

The method of any one of claims 22 to 24 wherein the deamidated plant protein is spray dried or freeze dried.

The method of any one of claims 22 to 25 further comprising, applying one or more centrifugation or filtration to separate at least a portion of the deamidated plant protein from the aqueous solution prior to drying the deamidated protein.

The technology disclosed in this specification is further described by reference to the following examples, which are not intended to limit the full scope of the inventions disclosed in this specification.

### Example 1—Isolation of Plant Protein using Glutaminase Enzyem

Protein was isolated in one of two ways. The basic premise of the isolation was to increase the water solubility of protein in flour so that the protein dissolves and can be separated from other insoluble components of the flour. Two solubilization processes were used, one used alkaline solubilization the other used glutaminase. The glutaminase solubilization was run at different temperature. Otherwise, the processes were the same.

Process 1, alkaline solubilization, one part pea flour was dispersed in six parts water. Sodium hydroxide was used in sufficient amount and strength the raise the dispersion's pH 9. Pea flour was soaked in high pH dispersion for one hour one of two temperatures, room temperature (“RT”, about 21°) and 50° C. (the “solubilization step”). The high pH dispersion was centrifuged at 1000 times g for 10 minutes. The supernatant was retained and adjusted to pH 4.5 using sufficient HCI at sufficient strength and sat at room temperature for 1 hour after which the supernatant (with precipitated pea protein) was centrifuged at 5000 times g for 10 minutes. The protein curd (a pellet of solids collected at the bottom of the centrifuge tube) was redispersed, adjusted to neutral pH using sodium hydroxide and the dispersion was spray dried to collect a solid.

Process 2, alkaline solubilization, was the same as the alkaline solubilization except for the solubilization step. Process 2 was run two times using different temperatures for the solubilization step. In process 2, pea protein within the pea flour was solubilized by adding to the dispersed pea flour (1 part flour to 6 parts water) glutaminase enzyme (0.25% w/w protein/flour). Glutaminase reacted with the protein in the flour for 4 hours at one of two temperature 35° C. and 50° C. Note that there is no specific enzyme deactivation step. Enzyme is expected to be deactivated in the acidification step (pH 4.5, 60C for 1 hour) to recover the protein.

Pea protein isolate obtained using Process 1 (alkaline solubilization) and Process 2 (glutaminase solubilization) were evaluated for percent protein recovered, protein recovery efficiency, protein content of the protein isolate, and water solubility of the protein in the protein isolate at various pH.

The weight of the product (Wₚ) and the weight of the pea flour (W_{f}) were recorded. Protein content of the product (% Pₚ) and protein content of the pea flour (% P_{f}) were measured following AACC 46-30.01 (Crude Protein—Combustion Method). The protein recovery yield was calculated as:

$$Protein Recovery Yield = \frac{Wp \times \% Pp}{Wf \times \% Pf} \times 100 \%$$

Protein recovery efficiency of the conventional alkaline process (Process 1) at room temperature was designated as 100%. Protein recovery efficiency of other processes was calculated as:

$$\frac{Protein Recovery Yield}{Protein Recovery Yield of alkaline process at room temperature} \times 100 \%$$

Protein content of the isolate was measured following AACC 46-30.01 (Crude Protein—Combustion Method).

Protein solubility of the protein isolate samples was measured as following. The samples were dispersed in pH 3, pH5, or pH 7 buffers at 2% (w/v) concentration and mixed at room temperature for 1 hour to ensure complete hydration and dispersion of the proteins. The solution was then centrifuged at 5,000 g for 15 min. The weight of the supernatant (Wₛ) and the weight of the precipitate (Wₚ) were recorded. Protein content of the supernatant (% Proteinₛ) and protein content of the precipitate (% Proteinₚ) were measured following AACC 46-30.01 (Crude Protein Combustion Method). The protein recovery yield was calculated as:

$$Protein Solubility = ( {W}_{s} \times \% {Protein}_{s} ) / ( {W}_{s} \times \% {Protein}_{s} + {W}_{P} \times \% {Protein}_{P} ) \times 100 \%$$

Percent protein recovered and protein recovery efficiency are reported in Table 1.

TABLE 1
Protein Recovery: Alkaline vs. Glutaminase Solubilization

| | Percent Protein | Protein Recovery |
|---|---|---|
| | Recovered | Efficiency |
| Sample 1 - alkaline @ RT | 67.6 | 100% |
| Sample 2 - alkaline @ 50° C. | 65.1 | 96.4% |
| Sample 3 -glutaminase @ 50° C. | 60.9 | 90.1% |
| Sample 4 - glutaminase @ 35° C. | 63.8 | 94.4% |

Sample 1 was measured to have protein content 88.0% (wt. %, dry basis). Sample 4 was measured to have protein content of 87.1% (wt. %, dry basis).

Water solubility at various pH is reported in Table 2 for Samples 1 and 4.

TABLE 2
Water Solubility: Alkaline vs Glutaminase Solubilization

| | Alkaline at RT | Glutaminase @ 35° C. |
|---|---|---|
| % Protein Solubility at pH 3 | 9.0 | 15.6 |
| % Protein Solubility at pH 5 | 8.4 | 39.9 |
| % Protein Solubility at pH 7 | 12.1 | 49.1 |

What is claimed:

1. A method of isolating a plant protein comprising:
a) soaking a proteinaceous plant based starting material, in an aqueous solution comprising from about 0.01 to about 0.25% glutaminase enzyme (wt. % relative to the starting material) at a temperature less than about 45° C., for time of less than about 5 hours;
b) separating the deamidated plant protein from the aqueous solution; and
c) drying the deamidated plant protein.

2. (canceled)

3. The method of claim 1 wherein the proteinaceous plant-based material is selected from the group consisting of fava bean, pea, lentil, chickpea, and quinoa.

4. (canceled)

5. The method of claim 1 wherein the separating step comprises adjusting the pH of the aqueous solution to from about 4.0 to about 4.5.

6. The method of claim 1 wherein the separating step comprises adding calcium or a calcium containing compound to the aqueous solution.

7. (canceled)

8. (canceled)

9. The method of claim 1 further comprising a step following step a) comprising inactivating the glutaminase enzyme by increasing the temperature the aqueous solution comprising the pea flour to a temperature of at least about 80° C. for at least about 10 minutes.

10. The method of claim 1 wherein the separating step comprises, applying one or more centrifugation or filtration to separate at least a portion of the deamidated plant protein from the aqueous solution and then doing one or both of
a) adjusting the pH of the aqueous solution to from about 4.0 to about 4.5 and
b) adding calcium or calcium composition to the aqueous solution

11. The method of claim 1 wherein the separating step comprises
a) doing one or both of adjusting the pH of the aqueous solution to from about 4.0 to about 4.5, and adding calcium to the aqueous solution, and then
b) applying one or more of centrifugation or filtration to separate at least a portion of the plant protein from the aqueous solution.

12. (canceled)

13. The method of claim 1 wherein the deamidated plant protein is a deamidated pea protein having a degree of deamidation of between about 10% and about 20%.

14. The method of claim 1 wherein the deamidated plant protein is a deamidated pea protein having a percent soluble material content at pH 6 of greater than 30%.

15. (canceled)

16. (canceled)

17. (canceled)

18. (canceled)

19. The method of claim 1 having a yield (isolate recovered/protein in base flour×100) greater than about 60%.

20. The method of claim 1 wherein the method has a protein recovery efficiency of at least about 90%, compared with the conventional alkaline protein isolation process.

21. The method of claim 1 wherein the deamidated plant protein isolate or deamidated pea protein isolate has protein content (wt. %) from about 80% to about 90%.

22. (canceled)

23. (canceled)

24. (canceled)

25. (canceled)

26. (canceled)
